(19) United States
(12) Patent Application Publication
(10) Pub. No.: US 2005/0228059 A1
(43) Pub. Date: Oct. 13, 2005

(54) Process for the preparation of hydrocarbons

(75) Inventors: Hans Peter Alexander Calis, CM Amsterdam (NL); Michiel Jan Groeneveld, CM Amsterdam (NL); Guy Lode Magda Maria Verbist, CM Amsterdam (NL)

Correspondence Address: Jennifer D Adamson;Shell Oil Company, Intellectual Property, P O Box 2463, Houston, TX, 77252-2463, US

(73) Assignee: Shell Oil Company, Houston, TX (US)

(21) Appl. No.: 10/519,341

(22) PCT Filed: Jun. 18, 2003
(86) PCT No.: PCT/EP03/06449

§ 371 Date: Dec. 22, 2004

(30) Foreign Application Priority Data
Jun. 26, 2002 (EP) 02254499.3

(51) Int. Cl. C07C 27/26

(52) U.S. Cl. 518/726

(57) ABSTRACT

The invention concerns a process for the preparation of hydrocarbons and the generation of heat involving i) introducing a gas comprising carbon monoxide and hydrogen into a first reactor section having catalyst and introducing cooling fluid into this first reactor section; ii) allowing a part of the carbon monoxide to react catalytically in the first reactor section to hydrocarbons and water, at least part of the reaction heat being absorbed directly by the cooling-fluid; iii) withdrawing from the reactor section a stream consisting of the reaction product containing the hydrocarbons, water, unconverted feed and cooling fluid; iv) cooling down at least a part of the withdrawn stream containing cooling fluid to generate heat; v) optionally removing water from the withdrawn stream, and, vi) repeating at least once steps I-v with the stream obtained in step v in further reactor section(s). The invention further relates to a reactor suitable for carrying out the above process.

The present invention relates to a process for the preparation of hydrocarbons and the generation of heat by reaction of carbon monoxide and hydrogen in the presence of a catalyst at elevated temperature and pressure in at least two stages.

Many documents are known describing processes for the conversion of hydrocarbonaceous feedstocks, in particular gaseous hydrocarbonaceous feedstocks, especially methane from natural sources, e.g. natural gas, associated gas and/or coal-bed methane, into liquid products, especially oxygenates, e.g. DME and methanol, and liquid/solid hydrocarbons.

In several recent documents reference is made to abundant gaseous hydrocarbon feedstocks as natural gas and/or associated gas, at remote locations (e.g. in the dessert, tropical rain forest) and/or off-shore locations, where no direct use of the gas is possible, usually due to the absence of large human populations and/or the absence of any industry. Transportation of the gas, e.g. through a pipeline or in the form of liquefied natural gas, requires extremely high capital expenditure or is simply not practical. This holds even more in the case of relatively small gas production rates and/or gas fields. Reinjection of the gas (and production at a later moment) is another possibility, however, this will add to the costs of the production, and may, in the case of associated gas, result in undesired effects on the crude oil production. Burning of associated gas has become an undesired option in view of depletion of hydrocarbon sources and air pollution. Gas found together with crude oil is known as associated gas, whereas gas found separate from crude oil is known as non-associated gas or natural gas. Associated gas may be found as “solution gas” dissolved within the crude oil, and/or as “gas cap gas” adjacent to the main layer of crude oil. Associated gas is usually much richer in the larger hydrocarbon molecules (ethane, propane, butane) than non-associated gas.

A process often used for the conversion of hydrocarbonaceous feedstocks into liquid and/or solid hydrocarbons is the Fischer Tropsch process. The hydrocarbonaceous feedstock is converted in a first step into a mixture of hydrogen and carbon monoxide (often referred to as synthesis gas). The mixture of hydrogen and carbon monoxide is then converted in a second step over a suitable catalyst at elevated temperature and pressure into paraffinic compounds ranging from methane to high molecular weight molecules comprising up to 200 carbon atoms, or, under particular circumstances, even more.

Numerous catalysts have been used in carrying out the Fischer Tropsch reaction. Saturated as well as unsaturated compounds can be made, mainly depending on the catalytic metal compound, the use of one or more specific promoters, and reaction conditions as temperature, pressure, GHSV, H₂/CO ratio etc. The reaction is very exothermic and temperature sensitive whereby temperature control is required to maintain a desired hydrocarbon product selectivity.

Numerous types of reactor systems have been used for carrying out the Fischer Tropsch reaction. The developed Fischer Tropsch reactor systems include fixed bed reactors, especially multitubular fixed bed reactors, fluidised bed reactors as entrained fluidised bed reactors and fixed fluidised bed reactors, and slurry bed reactors as three phase slurry bubble columns and ebulated bed reactors.

The commercial fixed bed Fischer Tropsch reactor usually comprises a vertical, multitubular fixed bed reactor. Small catalyst particles (typically having a length of less than 15 mm in the characteristic diameter, the characteristic diameter usually around 1 to 3 mm) are packed in large amounts of long tubes (usually 8-16 m long), e.g. 1,000 to 10,000 tubes or even more, in a cylindrical vessel. Gas is usually introduced at the top of the tubes and product and any unconverted feed are collected at the end of the tubes. The tubes are surrounded by cooling medium, usually a mixture of water and steam. The catalyst bed typically contains voidages in the order of about 0.3 to 0.5 depending upon the specific particle shape (cylinders, trilobes, spheres etc.). Fixed bed reactors offer simplicity and conversion kinetics that are easy to scale up.

Fixed bed Fischer Tropsch reactors are often constrained by pressure drop and heat transport limitations. In general, high productivity and high C₅+ selectivities as well as low methane selectivities can generally be achieved with small catalyst particles, typically in the order of less than 200 microns. In this context, “selectivity” refers to the following ratio: (moles of referenced product formed)/(mole of CO converted). In fixed-bed reactor systems, however, the pressure drop limits the practical application to much larger catalyst particle sizes. Shaped extrudates (trilobes, quadralobes, etc.) in the range of 1 to 3 mm diameter are frequently used. Smaller size extrudates are hardly used because they are difficult to manufacture in commercial quantities and create high pressure drops across the bed.

The Fischer Tropsch reaction is characterized by a very high heat of reaction. Unfortunately, the heat transfer characteristics of fixed-bed reactors are generally poor because of the relatively low mass velocity. If one attempts, however, to improve the heat transfer by increasing the gas velocity, a higher CO conversion can be obtained but there is an excessive pressure drop across the reactor, which limits commercial viability. In order to obtain the CO conversions desired and gas throughputs of commercial interest, the needed conditions result in a high radial temperature profile. For that reason, the Fischer-Tropsch fixed-bed reactor diameter should be less than 5 or 7 cm to avoid these excessive radial temperature profiles. The desired use of high-activity catalysts in Fischer-Tropsch fixed-bed reactors, makes the situation even more worse. The poor heat transfer characteristics makes local run aways possible (hot spots), which may result in local deactivation of the catalyst. Often an axial temperature profile exist over the tube. As a certain maximum temperature cannot be exceeded, part of the catalyst works at a sub-optimum level.

As indicated above, the use of catalyst particle sizes greater than 200 micron diameter to avoid excessive pressure drop through the reactor results in high methane selectivity and low selectivities toward the high molecular weight paraffins, which generally have more economic value. This selectivity is due to a disproportional catalyst pore diffusion limitation on the rate of transport of reactants (CO and H₂) into the interior of the catalyst particle. To address the situation, the use of catalysts particles having the active metal component restricted to a thin layer on the outer edge of the particle has been suggested. These catalysts appear costly to prepare and do not appear to make good use of the available reactor volume.

The use of liquid recycles as a means of improving the overall performance in a fixed-bed design has been described. Such a system is also called a “trickle bed” reactor (as part of a subset of fixed-bed reactor systems) in which both reactant gas and an inert liquid are introduced (preferably in an upflow or down flow orientation with respect to the catalyst) simultaneously. The presence of the flowing reactant gas and liquid improves the reactor performance with respect to CO conversion and product selectivity. A limitation of the trickle bed system (as well as of any fixed-bed design) is the pressure drop associated with operating at high mass velocities. The gas-filled voidage in fixed-beds (typically <0.50) does not permit high mass velocities without excessive pressure drops. A too high a pressure drop can cause particle attrition/crushing. Consequently, the mass throughput undergoing conversion per unit reactor volume is limited due to the heat transfer rates. Increasing the individual catalyst particle size may slightly improve heat transfer by allowing higher mass velocities (for a given pressure drop), but the loss in selectivity toward the high boiling point products and the increase in methane selectivity combined with the increase in catalyst activity generally offset the commercial incentives of higher heat transfer.

The Fischer-Tropsch catalyst performance is sensitive to mass transfer limitations within the individual catalyst particles. It is known that Fischer-Tropsch product selectivity is sensitive to the H₂/CO feed ratio. Increasing this ratio leads to poor selectivity (i.e. high methane and lower boiling point liquids), but the catalyst productivity, which may be indicated by the expression: (volume CO converted)/(volume of catalyst-hour), increases. In fixed-bed operations that employ large catalyst particles with relatively long diffusion lengths, the H₂/CO ratio within the catalyst volume can change significantly. Consequently when utilizing larger catalyst particles to mitigate pressure drop and improve the heat transfer (through increasing mass velocity), the performance of the Fischer-Tropsch fixed-bed catalyst systems may degrade due to longer intra-particle diffusion distances resulting in increasing H₂/CO ratios, especially in the top parts of the bed. This degradation influences performance through lower productivities and lower selectivities towards higher-valued products.

Fischer-Tropsch three-phase slurry bubble column reactors generally offer advantages over the fixed-bed design in terms of heat transfer and diffusion characteristics. Numerous designs have been described that incorporate small catalyst particles suspended by the upflowing gas in a liquid continuous matrix. In this design, reactor diameters are no longer limited by heat transfer characteristics. The motion of the continuous liquid matrix allows sufficient heat transfer to achieve a high commercial productivity. The catalyst particles are moving within a liquid continuous phase, resulting in high heat transfer from the individual particles, while the large liquid inventory in the reactor provides a high degree of thermal inertia, which helps prevent rapid temperature increases that can lead to thermal runaway. Further, the small particle size minimizes the negative impact of diffusional resistances within the interior of the catalyst.

The major technical issues associated with three-phase bubble columns include hydrodynamics and solids management. Reactor parameters should be selected to allow sufficient gas/liquid contacting to achieve the desired CO conversion levels. In this reactor type the H₂ and CO reactants should transfer from the feed gas (bubbled into the reactor volume) into the liquid phase. Once in the liquid phase, the dissolved reactants contact the catalytic surface to undergo reaction. The transfer of reactants from the liquid phase to the catalyst surface depends upon the turbulence of the liquid continuous phase and the diffusional length to the catalytic surface. Smaller catalyst particles are preferred in slurry reactors to avoid mass transfer limitations that lead to unacceptable product selectivity.

Liquid-phase back-mixing, however, which is reported to be a strong function of reactor diameter, can result in a much lower kinetic driving force that requires more reactor volume than a fixed-bed reactor operating at the same conversion. The need to have sufficient gas-liquid-solid mixing and liquid-solid separation complicates the equipment requirements and scale-up issues associated with commercial designs.

Small particles can be used in these systems because they are readily fluidised by the gas flow. The pressure drop across the reactor is limited to approximately the static head of the bed. Small particles, because of their large surface area also result in improved liquid-solid mass transfer compared to fixed-bed Fischer-Tropsch hydrocarbon synthesis reactors. Ultimately, the particle size is limited by the solids management system.

With respect to the latter, solids management issues, there are a number of issues that complicate slurry reactor systems. First, the gas distributor itself can be a major issue. A distributor is desired that distributes in a more or less uniform manner across a potentially very large diameter while preventing “dead” zones in which the catalyst can settle out/down and lay on the reactor bottom. The reactor bottom itself may be the distributor. Second, catalyst/wax separation can be a significant technical hurdle, which limits minimum catalyst particle size and can be very negatively impacted by catalyst particle attrition—especially over long time periods and/or in concert with poorly designed gas distributors.

Commercial designs of fixed-bed and three-phase slurry reactors typically utilize boiling water to remove the heat of reaction. In the fixed-bed design, the individual reactor tubes are located within a jacket containing water/steam. The heat of reaction raises the temperature of the catalyst bed within each tube. This thermal energy is transferred to the tube wall forcing the water to boil within the jacket. In the slurry design, tubes are typically placed within the slurry volume and heat is transferred from the liquid continuous matrix to the tube walls. The production of steam within the tubes provides the needed cooling. The steam in turn is cooled/condensed in another heat exchanger outside of the reactor or used, optionally after superheating, to drive a steam turbine.

Fluidised bed type Fischer-Tropsch reactors also give much better heat transfer characteristics than fixed bed reactors and can employ very small catalyst particles. These reactors operate essentially “dry”, which means that the production rates of species which are liquid at reactor conditions must be very low, approaching zero. Otherwise, rapid catalyst defluidization can occur. In practice, this requires very high reactor operating temperatures, which typically lead to high selectivities to methane and the production of a number of less desirable chemical species, such as aromatics. Catalyst/gas separation can also be a significant technical and economic hurdle with fluidised bed systems.

A reactor system has been proposed in PCT Application WO 98/38147 that uses a parallel-channel monolithic catalyst support to provide a fixed, dispersed catalyst arrangement. The embodiments discussed and presented include a catalyst with elongated monolithic support (e.g. 10 cm axial length) with active metals incorporated into lengthwise channels. The application contemplates using this catalyst in a Taylor flow regime. “Taylor flow regime” typically signifies a small capillary flow having a large axial dimension compared to the effective radial dimension, e.g. L/D>1000. A Taylor flow of gas and liquid in a channel may be defined as periodic cylindrical gas bubbles in the liquid having almost the same diameter as the channel and without entrained gas bubbles between successive cylindrical bubbles.

An object of the present invention is to provide an efficient, low cost, compact process scheme to overcome the disadvantages of the above described processes for the production of especially normally liquid hydrocarbons from gaseous hydrocarbonaceous feedstocks. More especially the process of the invention relates to a process which converts the feedstock in a very high selectivity into the desired hydrocarbons. Associated with the very high selectivity, a very high thermal efficiency is obtained. Using the process of the invention, C₅+ carbon efficiencies of more than 90% can be obtained, while thermal efficiencies, for a fully optimised process, of above 75% can be obtained. In the present invention, it is proposed to carry out the Fischer Tropsch reaction in two or more, preferably adiabatic, reactor sections, each reactor section comprising a, preferably high voidage, fixed catalyst bed, in which reactants and cooling medium are introduced into the reactor sections, the reactants being partly converted, the cooling medium directly absorbing the heat generated in the Fischer Tropsch reaction. The reaction products, unconverted feed and heated cooling medium are withdrawn from the reactor sections, unconverted feed is, at least partly, reintroduced into (another) one of the reactor sections, hydrocarbon products may be withdrawn, water formed in the Fischer Tropsch reaction is preferably removed and heated cooling medium is cooled down under the simultaneous generation of heat and reintroduced into the reactor sections. Preferably hydrogen is added to the reactants between the reactor sections.

The present invention therefore relates to a process for the preparation of hydrocarbons and the generation of heat by reaction of carbon monoxide and hydrogen in the presence of a catalyst at elevated temperature and pressure in at least two stages, the process comprising:
i) introducing a gas comprising carbon monoxide and hydrogen into a first reactor section comprising catalyst and introducing cooling fluidum into this first reactor section;
ii) allowing a part of the carbon monoxide and hydrogen to react catalytically in the first reactor section to hydrocarbons and water, at least part of the reaction heat being absorbed directly by the cooling fluidum;
iii) withdrawing from the reactor section a stream consisting of the reaction product comprising the hydrocarbons, water, unconverted feed and cooling fluidum;
iv) cooling down at least part of the withdrawn stream comprising cooling fluidum to generate heat;
v) optionally removing water from the withdrawn stream;
vi) introducing stream obtained in step v) comprising at least unconverted carbon monoxide and hydrogen into a second or further reactor section comprising catalyst and introducing cooling fluidum into this second or further reactor section;
vii) optionally introducing a hydrogen containing stream into the second or further reactor section;
viii) allowing a part of the carbon monoxide and hydrogen to react catalytically in the second or further reactor section to hydrocarbons and water, at least part of the reaction heat being absorbed directly by the cooling fluidum;
ix) optionally repeating steps iii-viii in further reactor sections and
x) withdrawing from the last reactor section the reaction product comprising the hydrocarbons, water, any unconverted carbon monoxide, any unconverted hydrogen and cooling fluidum.

An important advantage of the proposed process is the possibility to reach very high CO conversion levels and very high C₅+ selectivities. Further, when compared with the usual fixed bed reactors, a product is obtained in which the amount of olefins is relatively high. This makes the product more useful for chemical applications. The relatively low pressure drop avoids the use of a large (and expensive) compressor. No gas recycle is needed to obtain the high conversion. The scale up of the fixed bed reactor is relatively easy. Catalyst loading and unloading is fairly simple when compared with the traditional fixed bed reactor. Introduction of structured catalysts, e.g. monolithic structures or plate structures covered with a thin layer of catalyst can easily be done. Optimum use can be made of the catalyst in view of the relatively short reactor beds, resulting relatively flat temperature profiles. The use of the cooling fluidum results in much improved heat transfer characteristics when compared with traditional fixed bed reactors. The use of a number reactor sections makes it possible to adapt the total process in several ways, for instance different catalysts can be used in different reactor sections, while the temperature of each reactor sections can be controlled in an independent way. Further, the catalyst may differ in size in each reactor section to use the total reactor space as efficient as possible. The removal of water between the stages allows higher reactant partial pressures (at the same total pressure), and results in less carbon dioxide formation. The potential addition of hydrogen between the stages makes it possible to use low H₂/CO ratios, resulting in high selectivities to C₅+ hydrocarbons. The absence of cooling internals in the reactor sections makes the construction of the reactor relatively easy and relatively cheap. Furthermore, no expensive reactor space is needed for an indirect cooling system. Standard heat exchange equipment can be used for cooling down the cooling fluidum. No expensive tube sheets are necessary. The problems encountered with slurry systems as scale up, kinetics control, back mixing, gas distribution and solids management do not exist. Much higher conversion and selectivities are obtained.

The number of stages (or reactor sections) is at least 2, preferably at least 3 in order to obtain a minimum of the above the described advantages. The maximum number may be up to 50 or even higher, but in order to make the process (and all hardware involved) and process control not to complicated, a number of at most 40 stages is preferred. Very suitably, to combine the optimum advantages of the new process and a not too complicated process and process control, the number of stages is between 5 and 20, more preferably between 8 and 12. In principle each reactor section can be operated in one reactor. It is preferred to combine several sections in one rector. Suitably at least 2 sections are combined in one reactor, while at most 25 reactor sections, preferably at most 15, are combined in one reactor. Too many reactor sections will result in more complicated hardware and process control. More preferably between 3 and 7 sections are combined in one reactor.

The H₂/CO (molar) ratio of the feed gas to the first reactor section may be between 3 and 0.3 or higher or lower. Very suitable the H₂/CO ratio is between 2.0 and 0.4, especially between 1.6 and 0.4, preferably between 1.1 and 0.5. It will be appreciated that lower H₂/CO ratios result in higher C₅+ selectivities. Thus low ratios are preferred. As the consumption ratio is usually between 2.0 and 2.1, the use of a feed ratio below the consumption ration will result in a decrease of the H₂/CO ratio during the reaction. It is desired that the ratio does not fall below 0.2, in order to avoid undesired side reactions, especially the formation of coke on the catalyst. In a preferred embodiment, the feed ratio to each reactor section is below the consumption ration, e.g. between 1.1 and 0.5, and hydrogen is added between the stages to increase the ratio again to a higher value, preferably to a value between 1.6 and 0.4, more preferably between 1.1 and 0.5. Hydrogen is preferably added as substantially pure hydrogen (i.e. more than 98 vol % hydrogen). However, also synthesis gas having a (very) high H₂/CO ratio may be used. For instance, a ratio of 4 may be used, preferably 6, more preferably 10. The hydrogen containing gas preferably does not comprise any inert gases (nitrogen, methane, noble gases etc.). The amount of inerts is preferably less than 10 vol %, more preferably less than 4 vol %.

The CO conversion per stage is suitably between 2 and 50 vol %, preferably between 3 and 40 vol %, more preferably between 6 and 15 vol % (conversion of CO based on feed stream to the first reactor section). It will be appreciated that the conversion per stage will be related to the total number of reactor section. For instance at a number of sections between 8 and 12, the CO conversion per stage will be between 12.5 and 8.3 vol %.

The process of the invention is suitably carried out in such a way that in the first reactor section, preferably all reactor sections, at least 50%, especially at least 80%, of the heat generated by the reaction is directly absorbed by the cooling fluidum, preferably at least 90%, more preferably at least 95%. Part of the heat may be removed by indirect cooling by means of a cooling system in the reactor section. This, however, is not a preferred embodiment. Additional indirect cooling may be used in a particular part of the section, in order to suppress the temperature locally, e.g. to avoid a particular maximum in the heat profile over the section.

The process of the invention as carried out in the separate reactor sections is preferably an adiabatic process, i.e. no heat is removed within the reactor sections. It will be appreciated that a small amount of reaction heat will dissipate via the walls of the reactor. This will be small with respect to the total amount of heat generated. More specifically, at least the first reactor section is an adiabatic reactor section, preferably all reactor sections are adiabatic reactor sections.

The temperature increase of the cooling fluid per reactor section is suitably between 3 and 30° C., preferably between 5 and 20° C., more preferably between 7 and 15° C. At lower levels the process will be less efficient, at higher levels the temperature difference between the entrance and the end of the catalyst bed will become to high. A too high temperature at the end may result in a decrease of C₅+ selectivity, and in some cases even catalyst deactivation, a too low temperature at the entrance of the bed results in less efficient use of the catalyst.

The process of the invention is suitably carried out at a GHSV of the carbon monoxide and the total hydrogen together between 2000 and 20000 Nl/l/h, preferably between 3000 and 10000 Nl/l/h based on total catalyst volume (including voids). The above feed stream comprises the feed to the first reactor section, as well as the intermediate hydrogen additions, inclusive any carbon monoxide when present. It does not comprise any inerts (methane, nitrogen, steam, etc.).

The process according to the invention usually uses a volume ratio (STP) between the gas fraction and the cooling fluidum fraction introduced in each reactor section is between 0.3 and 3, preferably 0.5 and 2, more preferably about 1. A lower value results in insufficient cooling capacity, a higher ratio will result in a too large amount of cooling fluid, which makes the reaction less efficient.

The catalyst to be used in the present process comprises suitably one or more metals active in the Fischer Tropsch reaction. Very suitable are iron, cobalt or nickel on a carrier, especially cobalt, preferably in combination with one or more promoters. The amount of catalytically active metal on the carrier (calculated as pure metal) is preferably in the range of from 3 to 300 pbw per 100 pbw of carrier material, more preferably from 10 to 80 pbw, especially from 20 to 60 pbw. The promoters may be selected from one or more metals or metal oxides. Suitable metal oxide promoters may be selected from Groups IIA, IIIB, IVB, VB and VIB of the Periodic Table of Elements, or the actinides and lanthanides. In particular, oxides of magnesium, calcium, strontium, barium, scandium, yttrium, lanthanum, cerium, titanium, zirconium, hafnium, thorium, uranium, vanadium, chromium and manganese are most suitable promoters. Particularly preferred metal oxide promoters for the catalyst used to prepare the waxes for use in the present invention are manganese and zirconium oxide. Suitable metal promoters may be selected from Groups VIIB or VIII of the Periodic Table. Rhenium and Group VIII noble metals are particularly suitable, with platinum and palladium being especially preferred. The amount of promoter present in the catalyst is suitably in the range of from 0.01 to 100 pbw, preferably 0.1 to 40, more preferably 1 to 20 pbw, per 100 pbw of carrier.

The process of the invention suitably uses a catalyst system in the form of a fixed bed, preferably a fixed bed having a void volume between 50 and 85 vol %, preferably between 60 and 80 vol %. In principle any shape of the catalyst is possible. Spheres, hollow spheres, extrudates, hollow extrudates, rings, saddles, structured packings etc. are possible. In order to reach the preferred void volumes, the fixed bed comprises preferably one or more monolithic structures, preferably ceramic monolithic structures, metal extruded monolithes or carbon monolithes, layers of corrugated plates, especially metal corrugated plates, gauzes, especially metal gauzes or shavings, especially metal shavings. The ceramic carrier is suitably a porous refractory oxide, preferably selected from silica, alumina, titania, zirconia. In another embodiment the carrier is a plate, gauze or shaving made from aluminium, iron or copper, especially stainless steel. It will be appreciated that the all reactor sections may comprise the same catalyst, but also that different reactor sections may contain different catalysts. Depending on the exact composition of the feed for a certain reactor section and the objective to be met by the specific reactor section, a different catalyst may be used. In addition, depending on feed, catalyst and objective specific reaction conditions may be used in the reactor sections.

The cooling fluidum to be used in the process of the invention suitably consists of one or more organic compounds, preferably Fischer Tropsch hydrocarbons, more especially C₁₄+ Fischer Tropsch hydrocarbons. It will be appreciated that at the start of the reaction a certain fluidum may be used, however, when the cooling fluidum is used in a recirculating process, which is a preferred embodiment, the starting cooling fluidum will be removed from the reaction together with the liquid reaction product, and gradually the cooling fluidum will be replaced by Fischer Tropsch liquid product. It will be appreciated that the cooling fluidum is preferably inert and stable during the reaction conditions.

In the present process, heat is exchanged in such a way that the temperature of the stream cooling fluidum withdrawn from any reactor section and to be introduced in another section is decreased by 5-20° C., preferably 7-15° C., more preferably by the temperature increase of the reactor section involved. In that way a stable process is obtained. In specific circumstances, the amount of heat exchange is adjusted in such a way that a temperature profile is created over the all reactor sections, preferably a continuous temperature increase over all reactor sections. Also depending on specific catalysts more or less heat may be exchanged in order to create the desired temperature in each reactor section.

Suitably, the stream withdrawn from a reactor section is separated into a liquid stream and a gaseous stream, followed by cooling down the liquid stream and cooling down the gaseous stream, suitably to a temperature between 80 and 150° C., preferably to a temperature between 90 and 130° C. The liquid stream comprises liquid reaction product and cooling fluidum, the gaseous product comprises unconverted reactants, gaseous hydrocarbon products, steam and, if present, inerts. It will be appreciated that it is also possible to first cool down the stream withdrawn from a reactor section followed by separation into a liquid stream and a gaseous stream, followed by cooling down the gaseous stream. Also combinations are possible. The cooled down liquid stream is used as cooling fluidum in the same or another reactor section. Part of the cooled down product comprising the liquid product is to be removed from the process as the desired product, or is sent to a further work-up section. As in most cases the cooling fluidum will be the same as the reaction product, there is no need to separate between cooling fluidum and reaction product. The gaseous stream is cooled down suitably to a temperature between 80 and 150° C., preferably to a temperature between 90 and 130° C. Cooling down the gaseous stream results in the condensation of hydrocarbons and water. The water is preferably separated from the condensation product, the hydrocarbon stream will leave the process as the desired product, or is sent to a further work-up section. It is less desired to use the condensed product as cooling fluidum in one or more reactor sections, as a large amount will evaporate under the reaction conditions, resulting in a decrease of reactant partial pressures. The remaining gaseous stream, comprising at least unconverted feed, is introduced into the following reactor section. Preferable additional hydrogen is used to this stream. For efficiency reason, there is the possibility to combine the gaseous streams from two or more reactors, followed by cooling down. Please note that it is also possible to use two or more equivalent reactors comprising equivalent reactor sections, and to combine withdrawn stream from equivalent reactor section, followed by combined cooling and further processing, after which the gaseous streams and cooling fluidum are reintroduced in the following equivalent reactor sections. Similar constructions are also possible for the cooling down of the liquid streams, as well in one reactor comprising several reactor sections, as well as between two or more equivalent reactors. Part or all water may also be removed by means of membrane separation from the withdrawn gaseous streams.

Preferably the amount of water which is removed from the withdrawn stream after a reactor section is between 50 and 95% of the water formed in the reaction, preferably between 60 and 90. This can be obtained by using the preferred temperature ranges as described above.

It will be appreciated that cooled down cooling fluidum from a reactor section may be introduced into the same reactor section or into a different reactor section. Suitably the cooled down cooling fluidum from a reactor section is introduced into the next reactor section. Further, cooling fluidum from a number of reaction sections may combined and re-introduced in a number of reactor sections. As the cooling fluidum absorbs most of the heat generated in a reactor section, it will be clear that the temperature control of a particular section can be realised by the amount of cooling fluidum sent to a particular reactor section and by the temperature of the cooling fluidum. The preferred option is the control of the amount, as this can be easily controlled.

In order to carry the process of the invention out in an efficient way, at least 75 vol % of the unconverted carbon monoxide and hydrogen from a reactor section is introduced into the next reactor section, preferably 90%, more preferably 100%.

The temperature of the hydrocarbon synthesis reaction is suitably between 170 and 320° C., preferably between 190 and 270° C., and the pressure is between 5 and 150 bar, preferably between 20 and 80 bar. The pressure drop between the inlet of a reactor section and the inlet of the consecutive reactor section is between 1000 and 50000 Pa, preferably between 5000 and 40000 Pa, more preferably between 10000 and 25000 Pa.

The process of the present invention is suitably carried out with a mixture of hydrogen and carbon monoxide without any inert gases. This results in the most efficient process. As, however, the pressure drop, when compared with the usual fixed bed reactors is considerable less severe, it is also possible to use synthesis gas containing a certain amount of inerts. Suitably the gas feed to the first reactor section may comprises up till 50 vol % inerts, preferably up till 20 vol %, more preferably up till 10 vol %. The inerts, especially nitrogen, may be present in the oxygen containing gas stream which is used in the partial oxidation of the hydrocarbonaceous feed, or may be present in the hydrocarbonaceous feed itself, for instance nitrogen and/or noble gases in natural gas.

The normally liquid hydrocarbons are especially mixtures of C₅-C₁₈ hydrocarbons, although small amounts of C₄− and C₁₉+ compounds may be present. At STP, these mixtures are liquid. C₁-C₄ compounds are considered as normally gaseous hydrocarbons. Normally solid hydrocarbons are especially mixtures of C₁₉+ compounds, up to C₂₀₀. Smaller quantities of C₁₈− may be present. Normally solid hydrocarbons are solid at STP. The hydrocarbon mixture made in the Fischer Tropsch process vary from C₁ to C₂₀₀ or even higher. The amount of C₁₉+ hydrocarbons is preferably at least 60 wt %, preferably 70 wt %, more preferably 80 wt %. These hydrocarbons are paraffinic in nature, although considerable amounts of olefins and/or oxygenates may be present. Suitably up to 20 wt %, preferably up to 10 wt %, of either olefins or oxygenated compounds may be present. The compounds are mostly normal compounds, although a few wt % of branched, especially methyl branched, may be present.

A part may boil above the boiling point range of the so-called middle distillates, but it might be desired to keep this part relatively small to avoid problems with respect to normally solid hydrocarbons. A most suitable catalyst for this purpose is a cobalt-containing Fischer-Tropsch catalyst. The term “middle distillates”, as used herein, is a reference to hydrocarbon mixtures of which the boiling point range corresponds substantially to that of kerosene and gas oil fractions obtained in a conventional atmospheric distillation of crude mineral oil. The boiling point range of middle distillates generally lies within the range of about 150 to about 360° C.

The higher boiling range paraffinic hydrocarbons obtained in the present process may be isolated and subjected to a catalytic hydrocracking, which is known per se in the art, to yield middle distillates. The catalytic hydrocracking is carried out by contacting the paraffinic hydrocarbons at elevated temperature and pressure and in the presence of hydrogen with a catalyst containing one or more metals having hydrogenation activity, and supported on a carrier. Suitable hydrocracking catalysts include catalysts comprising metals selected from Groups VIB and VIII of the Periodic Table of Elements. Preferably, the hydrocracking catalysts contain one or more noble metals from group VIII. Preferred noble metals are platinum, palladium, rhodium, ruthenium, iridium and osmium. Most preferred catalysts for use in the hydrocracking stage are those comprising platinum. To keep the process as simple as possible, the hydrocracking will usually not be a preferred option.

The amount of catalytically active metal present in the hydrocracking catalyst may vary within wide limits and is typically in the range of from about 0.05 to about 5 parts by weight per 100 parts by weight of the carrier material.

Suitable conditions for the catalytic hydrocracking are known in the art. Typically, the hydrocracking is effected at a temperature in the range of from about 175 to 400° C. Typical hydrogen partial pressures applied in the hydrocracking process are in the range of from 10 to 250 bar.

The invention further relates to one or more reactors for carrying out the process as described above. A very suitable reactor is an elongated cylindrical vessel, which, when in use, will be a vertical reactor. In one of the preferred embodiments, in which one reactor comprises 3 to 7 rector sections, the reactor will contain 2 to 6 plates, suitably at about the same distance, thus creating the 3 to 7 reactor sections. Also when in use, the 2 to 6 plates dividing the reactor in the several reactor sections, are preferably in a horizontal position. Each reactor section will contain a fixed catalyst bed, means for distributing gas and liquid over the catalyst bed at the upstream end of the catalyst bed and means for collecting gas and liquid at the downstream end of the catalyst bed. In a suitable design only one (large) catalyst bed will be present, delimited by the outside reactor wall. Above the catalyst bed will be a space for the distribution means, below the reactor bed there will be a space for collecting gas and liquid. Gas and liquid may be removed from the reactor section by one or more pipes for the liquid, and one or more pipes for the gas. In an alternative, gas and liquid may be removed via one or more common pipes, followed by separation (in one or more standard separation vessels) outside the reactor. Usually gas and cooling fluidum will be introduced into the top of the first reactor section above the catalyst bed. Gas and liquid will be removed from the reactor at the lower end of the first section, and, after separation, cooling, removal of liquid product and, often, water removal and optional addition of hydrogen, introduced into the top of the second section etc. As discussed above removal of water may be done after each reactor section, but also after each second, or even third, reactor section. Also, the liquid streams of several sections may be combined and cooled, followed by reintroduction in the reactor sections. Liquid from one section may be introduced may also be reintroduced into the same reactor section. The gas stream will in most cases flow from the first section to the second section, to the third section etc. Beside vertical reactors, it is also possible to use horizontal reactors. These horizontal reactors may comprise similar compartments as described for the vertical reactors, but may also comprise compartments with structured catalyst packings containing substantially horizontal channels through which a gas/liquid dispersion is transferred in horizontal direction.

1. A process for the preparation of hydrocarbons and the generation of heat by reaction of carbon monoxide and hydrogen in the presence of a catalyst at elevated temperature and pressure in at least two stages, the process comprising:
i) introducing a gas comprising carbon monoxide and hydrogen into a first reactor section comprising catalyst and introducing cooling fluid into this first reactor section;
ii) allowing a part of the carbon monoxide and hydrogen to react catalytically in the first reactor section to hydrocarbons and water, at least part of the reaction heat being absorbed directly by the cooling fluid;
iii) withdrawing from the reactor section a reaction product stream comprising the hydrocarbons, water, unconverted feed and cooling fluid;
iv) cooling down at least part of the withdrawn stream comprising cooling to fluid generate heat;
v) optionally removing water from the withdrawn stream;
vi) introducing stream obtained in step v) comprising at least unconverted carbon monoxide and hydrogen into a second or further reactor section comprising catalyst and introducing cooling fluid into this second or further reactor section;
vii) optionally introducing a hydrogen containing stream into the second or further reactor section;
viii) allowing a part of the carbon monoxide and hydrogen to react catalytically in the second or further reactor section to hydrocarbons and water, at least part of the reaction heat being absorbed directly by the cooling fluid;
ix) optionally repeating steps iii-viii in further reactor sections; and,
x) withdrawing from the last reactor section the reaction product comprising the hydrocarbons, water, any unconverted carbon monoxide, any unconverted hydrogen and cooling fluid.

2. The process of claim 1, in which the number of stages is between 5 and 20.

3. The process of claim 1, in which the CO conversion per stage is between 3 and 40 vol %.

4. The process of claim 1, in which the H₂/CO ratio of the gas feed to the first stage is between 1.6 and 0.4.

5. The process of claim 1, in which in the first reactor section, at least 50% of the heat generated by the reaction is directly absorbed by the cooling fluid.

6. The process of claim 5, in which the first reactor section is an adiabatic reactor section.

7. The process of claim 1, in which the temperature increase of the cooling fluid per reactor section is between 5° C. and 20° C.

8. The process of claim 1, in which GHSV of the carbon monoxide and hydrogen together is between 2000 and 20000 Nl/l/h; based on total catalyst volume.

9. The process of claim 1, in which the volume ratio between the gas fraction and the cooling fluid fraction introduced in each reactor section is between 0.5 and 2.

10. The process of claim 1, in which the catalyst comprises iron, cobalt or nickel on a carrier.

11. The process of claim 10, in which the catalyst comprises a carrier in the form of a fixed bed.

12. The process of claim 11, in which the fixed bed comprises one or more monolithic structures.

13. The process of claim 1, in which heat is exchanged to decrease the temperature of the stream withdrawn from any reactor section by 5-20° C.

14. The process of claim 1, in which the cooled down stream withdrawn from one or more reactor sections, is separated into a liquid stream and a gaseous stream, followed by further cooling down the gaseous stream, to a temperature between 80 and 150° C.

15. The process of claim 1, in which water is removed from the process by separating water from the withdrawn stream from the reactor sections/

16. The process of claim 1, in which cooled down cooling fluid from a reactor section is introduced into the same reactor section or in which cooled down cooling fluid from a reactor section is introduced into the next reactor section.

17. The process of claim 1, in which the temperature of the hydrocarbon synthesis reaction is between 170 and 320° C.

18. A reactor suitable for carrying out a process for the preparation of hydrocarbons and the generation of heat by reaction of carbon monoxide and hydrogen in the presence of a catalyst at elevated temperature and pressure in at least two stages, the process comprising:
i) introducing a gas comprising carbon monoxide and hydrogen into a first reactor section comprising catalyst and introducing cooling fluid into this first reactor section;
ii) allowing a part of the carbon monoxide and hydrogen to react catalytically in the first reactor section to hydrocarbons and water. at least part of the reaction heat being absorbed directly by the cooling fluid;
iii) withdrawing from the reactor section a reaction product stream comprising the hydrocarbons, water, unconverted feed and cooling fluid;
iv) cooling down at least part of the withdrawn stream comprising cooling fluid to generate heat;
v) optionally removing water from the withdrawn stream;
vi) introducing stream obtained in step v) comprising at least unconverted carbon monoxide and hydrogen into a second or further reactor section comprising catalyst and introducing cooling fluid into this second or further reactor section;
vii) optionally introducing a hydrogen containing stream into the second or further reactor section;
viii) allowing a part of the carbon monoxide and hydrogen to react catalytically in the second or further reactor section to hydrocarbons and water, at least part of the reaction heat being absorbed directly by the cooling fluid;
ix) optionally repeating steps iii-viii in further reactor sections; and,
x) withdrawing from the last reactor section the reaction product comprising the hydrocarbons, water, any unconverted carbon monoxide, any unconverted hydrogen and cooling fluid.

19. The process of claim 1, in which the number of stages is between 8 and 12.

20. The process of claim 1, in which the CO conversion per stage is between 6 and 15 vol %.

21. The process of claim 1, in which the H₂/CO ratio of the gas feed to the first stage is between 0.5 and 1.1.

22. The process of claim 1, in which additional hydrogen is introduced in the one or more stages following the first stage.

23. The process of claim 22, in which the H₂/CO ratio in the second and further stages is between 0.4 and 1.6.

24. The process of claim 22, in which the H₂/CO ratio in the second and further stages is between 0.5 and 1.1.

25. The process of claim 1, in which in all reactor sections at least 50% of the heat generated by the reaction is directly absorbed by the cooling fluid.

26. The process of claim 1, in which at least 90% of the heat generated by the reaction is directly absorbed by the cooling fluid.

27. The process of claim 1, in which all reactor sections are adiabatic reactor sections.

28. The process of claim 1, in which the temperature increase of the cooling fluid per reactor section is between 70° C. and 15° C.

29. The process of claim 1, in which GHSV of the carbon monoxide and hydrogen together is between 3000 and 10,000 Nl/l/h based on total catalyst volume.

30. The process of claim 1, in which the catalyst comprises cobalt on a carrier.

31. The process of claim 30, in which the catalyst further comprises one or more promoters selected from manganese and zirconium oxide or rhenium and platinum.

32. The process of claim 30, in which the catalyst comprises a carrier in the form of a fixed bed.

33. The process of claim 32, in which the fixed bed has a void ratio between 50 vol % and 85 vol %.

34. The process of claim 32, in which the fixed bed has a void ratio between 60 vol % and 80 vol %.

35. The process of claim 32, in which the fixed bed comprises a carrier in the form of a fixed bed.

36. The process of claim 10, in which the catalyst further comprises one or more promoters selected from manganese and zirconium oxide or rhenium and platinum.

37. The process of claim 11, in which the fixed bed has a void ratio between 50 vol % and 85 vol %.

38. The process of claim 11, in which the fixed bed has a void ratio between 60 vol % and 80 vol %.

39. The process of claim 12, in which the monolithic structures are selected from the group consisting of ceramic monolithic structures, metal extruded monoliths, carbon monoliths, layers of corrugated plates, gauzes and shavings.

40. The process of claim 1, in which heat is exchanged to decrease the temperature of the stream withdrawn from any reactor section by 7° C. to 15° C.

41. The process of claim 1, in which heat is exchanged to decrease the temperature of the stream withdrawn from any reactor section by the temperature increase of the reactor section involved.

42. The process of claim 1, in which the temperature of the hydrocarbon synthesis reaction is between 190° C. and 270° C. and the pressure is between 20 bar and 80 bar.
